(19) United States
(12) Patent Application Publication
(10) Pub. No.: US 2008/0177236 A1
(43) Pub. Date: Jul. 24, 2008

(54) LENGTH COMPENSATION FOR A SETTING MECHANISM OF AN INJECTION DEVICE

(76) Inventors: Stefan Burren, Bremgarten (CH); Ulrich Moser, Heimiswil (CH); Christian Schrul, Burgdorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/923,324

(22) Filed: Oct. 24, 2007

Related U.S. Application Data
(63) Continuation of application No. PCT/CH2006/000218, filed on Apr. 20, 2006.

(30) Foreign Application Priority Data
Apr. 25, 2005 (DE) 102005019428.1

(51) Int. Cl. A61M 5/315

(52) U.S. Cl. 604/224

(57) ABSTRACT

An adjusting device, and method, used to adjust a dose of a substance to be administered by an injection device, wherein the adjusting device includes a first element and at least one additional element displaceable relative to the first element, the first element having at least two thread pitches which are offset in relation to each other such that at least one thread element of the at least one additional element is only guided into one of the at least two thread pitches of the first element. The invention encompasses a method for making an adjusting device for an injection device, wherein the thread of at least one dosing element is introduced into an associated thread of an additional dosing element, such that a predetermined position ratio between the dosing element and the additional dosing element is established.

### CROSS-REFERENCE TO RELATED APPLICATIONS

This application is a continuation of International Application No. PCT/CH2006/000218, filed 20 Apr. 2006, which claims the priority from the German Application No. DE 10 2005 019 428.1, filed 25 Apr. 2005, the subject matter of both of which are hereby incorporated by reference in their entirety.

### BACKGROUND

The present invention relates to devices for injecting, infusing, administering, delivering or dispensing substances, and to methods of making and using such devices. More particularly, the present invention relates to a setting mechanism, in particular a dose setting mechanism, for setting a dose to be administered from an injection device or a pen. More particularly, it relates to a dose setting mechanism for setting one or more fixedly predefined dose units or for priming an injection device in readiness for administering one or more fixedly preset doses from the injection device and/or an ampoule which can be inserted in the injection device, and to a method of manufacturing the injection device and/or the dose setting mechanism.

A dose setting mechanism for an injection device is disclosed in German patent application 10 2005 001 159.4, and another mechanism of the type to which the present invention relates is known from patent specification WO 97/36626. The device comprises a housing with a reservoir for the product. Accommodated in the reservoir is a plunger, which forces the product out of the reservoir through an outlet of the reservoir when pushed in the forward feed direction. The plunger rod is a toothed rack which pushes the plunger in the forward feed direction. Also accommodated in the housing is a drive element which can be displaced relative to the housing in and opposite the forward feed direction and which drives the toothed rack. To this end, the drive element has drivers, which engage in rows of teeth of the toothed rack. To set the quantity of product administered with a stroke, i.e. when the dose setting mechanism is operated, the drive element is pulled back manually from a forward position, in the direction opposite the forward feed direction, by a set dose path length. As this happens, the drivers of the drive element slide across the teeth of the rows of teeth of the toothed rack and flex elastically. The toothed rack is prevented from being pushed back by locking means mounted so as to prevent any movement relative to the housing. The locking means co-operate with one of the rows of teeth of the toothed rack in such a way that the locking means prevent the toothed rack from moving opposite the forward feed direction. They flex elastically to permit a movement of the toothed rack in the forward feed direction. When the drive knob is operated, the drive element causes the toothed rack or plunger to move by the set dose path length so that the set dose is dispensed through the outlet of the reservoir.

One potential difficulty with such devices is that only slight manufacturing tolerances should occur and/or be tolerated because exactly set quantities have to be dispensed from injection devices, especially if the quantity is small.

### SUMMARY

One object of the present invention is to provide a setting or dose setting mechanism for an injection device which can be manufactured inexpensively.

In one embodiment, the present invention comprises an adjusting device, and method, used to adjust a dose of a substance to be administered by an injection device, wherein the adjusting device includes a first element and at least one additional element displaceable relative to the first element, the first element having at least two thread pitches which are offset in relation to each other such that at least one thread element of the at least one additional element is only guided into one of the at least two thread pitches of the first element. The invention encompasses a method for making an adjusting device for an injection device, wherein the thread of at least one dosing element is introduced into an associated thread of an additional dosing element, such that a predetermined position ratio between the dosing element and the additional dosing element is established.

For the purpose of the present invention, at least one component of a setting or dose setting mechanism and/or an injection device is provided with at least two and possibly three to twelve or more than twelve engaging elements or guides or threads, such as grooves, so that appropriate co-operating and/or complementary elements or threads of another component may be moved into or inserted in different positions, for example radially and/or axially offset positions. Since at least one component of a dose setting mechanism or injection device in accordance with the present invention is designed so that another component can be inserted in at least two different radially and/or axially offset fitted positions, it is possible to make a correction or compensation in length for individual parts which are of differing length due to manufacturing tolerances, for example, when the dose setting mechanism or the injection device is being assembled by fitting a component in one of several possible fitting positions.

In accordance with the present invention, it is no longer necessary for the individual parts of a dose setting or injection device to be manufactured with low tolerance windows because, as a result of the invention, individual parts can be assembled in a plurality of different orientations or relative positions depending on the respective actual shape and conforming to or deviating from a predefined norm, thereby compensating for manufacturing tolerances. In some preferred embodiments, e.g., in the case of a dose setting mechanism or injection device used for setting or dispensing small dose quantities, individual parts can be manufactured with greater or larger manufacturing tolerances, thereby reducing manufacturing costs, and the manufacturing tolerances can be compensated on the basis of the resultant manufacturing tolerance of the individual part when the individual parts are being assembled. This means that a dose setting or injection device can be manufactured which enables a precise dose to be set in a relatively small range, which can not be achieved with components with bigger manufacturing tolerances made by manufacturing methods known from the prior art.

By preference, in some embodiments of the present invention, at least one component of a dose setting mechanism or an injection device, such as a rotating sleeve, a housing and/or a forward feed element, is provided with two, three to twelve, or more than twelve threads which are radially and/or axially offset from one another. The threads are provided as circumferentially extending grooves on the internal face and/or external face of the respective component and may be offset from one another exactly or with small deviations of 360°/n, where n is a natural number greater than 1. For example, circumferentially extending grooves or threads may be offset from one another by 120° or 60°. The mutually offset threads may be disposed non-uniformly offset from one another around the component, in other words, with respect to the standard thread used in the case of an exact manufacturing process without any manufacturing tolerances, a thread maybe disposed offset to the side of the standard thread by a first angle and a second thread maybe disposed offset to the opposite side by a second angle that is different from the first angle, for example by±5° or±2°.

In some embodiments, the element of the dose setting mechanism or injection device which co-operates with an element having at least two mutually offset threads and which does not engage in all threads has at least one thread, or possibly two, three or more than three threads, which may be provided in the form of circumferentially extending webs projecting out from a thread base. In some embodiments of the present invention, two elements which may be displaced and/or rotated relative to one another are coupled by mutually engaging guides or threads so that one element can be fitted in the other element in at least two different relative positions or guide positions. For example, the external thread of a rotating sleeve can be fitted in one of at least two different mutually offset internal threads of a housing in the form of grooves so that, in the state of being fully screwed into the housing, a front end or a stop position of the rotating sleeve is positioned such that engaging elements or snappers provided on a front end of the housing are moved between the teeth of a toothed rack, guided in the injection device by a forward feed sleeve inserted in the rotating sleeve in one of several positions, when retained by snappers by the forward feed element mounted in the rotating sleeve, as a result of which snappers of the housing or snappers of the forward feed sleeve are prevented from being left in intermediate positions of the toothed rack or lying on them. However, if the snappers of the forward feed sleeve lie on the teeth of the toothed rack in a position defined by a stop, the rotating sleeve and/or the forward feed element can be fitted in another possible position to adjust this position.

In some embodiments, it may be preferable for the at least two mutually offset threads to be provided as threads of an internal thread or external thread on a first element of the injection device or dose setting mechanism which co-operate with one or a plurality of threads of another element disposed inside or outside the first element, for example, thereby permitting a relative movement guided by the mutually engaging thread or threads, e.g. a rotating movement.

Generally speaking, the expression “thread” as used in the context of the present invention should be understood as meaning not only a thread or thread pitch extending around an entire circumference, but also thread parts or thread pitch segments provided on only certain portions of an internal or external face of an element which are able to co-operate with a co-operating thread due to a thread web engaging in a thread groove, for example.

By virtue of another aspect of the present invention, the invention relates to a dose setting mechanism with at least one element of the type described above, having at least two mutually offset threads, and one thread provided in the form of a groove extending around the circumference or a portion of the circumference is moved into engagement with one of the at least two mutually offset threads of the element provided in the form of grooves. This provides that one thread is guided in a co-operating thread in the desired manner, and manufacturing tolerances can be compensated due to the possibility of selecting one of at least two possible options when screwing in the component to achieve a positional relationship of the components relative to one another predefined by a stop position.

In some preferred embodiments, the predefined positional relationship is set or defined on the basis of a distance which snappers or engaging elements of one component assume relative to snappers or engaging elements of another component coupled directly or indirectly with the component in a position defined by a front end position or a stop, for example.

The present invention also relates to a method of manufacturing a dose setting mechanism or an injection device, whereby the dose setting mechanism maybe provided as an integral component of the injection device or may comprise separate elements, and the dose setting mechanism has at least one setting element in the form of an inner element, for example, which is mounted in another element provided as an outer element and is guided by an external thread in an internal thread of the outer element for example, and for one thread of the inner or outer element, provided in the form of a web, for example, at least two threads are provided in the co-operating element in the form of circumferentially extending recesses or grooves designed to guide the thread, so that the inner element can be fitted on or screwed into the outer element in at least two different ways, thereby resulting in a predefined positional relationship between the inner element and the outer element so that a manufacturing tolerance can be compensated. In some embodiments, the predefined positional relationship may be fixed in a defined stop position due to the position of a snapper or engaging element provided, for example, on the inner element or outer element.

In accordance with the present invention, a dose setting mechanism for an injection device may have a setting element, which may be a cylindrical rotating sleeve or a knob which can be fixedly or rotatably connected to a cylindrical rotating sleeve. The rotating sleeve has at last two internal threads and external threads, in some preferred embodiments extending in the same direction and/or disposed coaxially with one another, so that the rotating sleeve has an internal thread and an external thread which overlap in at least one region and which may also be disposed around more or less the entire length of the rotating sleeve on the internal face and the external face of the cylindrical rotating sleeve body.

In some embodiments, the threads maybe designed as moving threads of the thread type not retained by friction, and may be threads, structures, elements or cams engaging in threads or circumferentially extending spirals which engage in or co-operate with the threads of oppositely lying co-operating threads respectively. Provided inside and outside the rotating sleeve are elements of the injection device which are secured so that they are not able to rotate relative to one another or which are secured so that they are not able to rotate relative to a housing of the injection device, and an element inside or outside the rotating sleeve lying adjacent to the latter may also be a part of the housing of the injection device. The elements lying inside and outside the rotating sleeve may themselves also be cylindrical, for example, or may extend around only one or more part regions in the circumferential direction of the rotating sleeve, and have co-operating threads in the form of individual cams or circumferentially extending spiral grooves or webs, for example, which are able to engage in the internal and external threads of the rotating sleeve or co-operate with appropriate thread elements of the rotating sleeve. This being the case, the internal thread of the rotating sleeve may have the same or a different pitch from the external thread of the rotating sleeve.

If, in some embodiments, a forward feed element of the injection device is mounted inside the rotating sleeve, for example, and is disposed in one specific engagement position of several possible thread engagement positions with the rotating sleeve, the forward feed element can be moved in a defined manner by a predefined distance in the proximal and distal direction of the injection device by rotating the rotating sleeve, which is in one specific engagement position of several engagement positions with a housing part of the injection device lying on the external face of the rotating sleeve, for example. If the internal thread of the rotating sleeve has a smaller pitch than the external thread of the rotating sleeve, when the rotating sleeve is screwed out of the housing of the injection device, with which the rotating sleeve sits in a thread engagement, by the axial length D for example, the forward feed element is moved in the same direction by a shorter distance d, thereby achieving a shorter setting movement based on a compact design. This being the case, a short functional distance d can be increased in ratio to a longer distance D, thus making it easy to set a fixed dose, for example.

In some embodiments, if a driver such as a catch element, for example, is joined to the forward feed element, for example disposed on a flexible element or arm of the forward feed element, a cam or lug provided on the flexible element and projecting radially inwardly, may be guided by means of the teeth of a toothed rack serving as a plunger rod or the thread of a threaded rod. Depending on the extent of the axial movement of the forward feed element relative to the toothed rack, one or more teeth of the toothed rack are able to move across the elastically mounted cam or lug, as a result of which one or more “clicking” noises are generated and the dose to be dispensed from the injection device is set. The dose set in this manner is dispensed due to a movement of the forward feed element or forward feed sleeve in the distal direction caused by pushing back or rotating the rotating sleeve, which is transmitted to the toothed rack due to an engagement of the cams or lugs and then to a stop lying directly or indirectly on the toothed rack which is pushed into an ampoule in order to force out a substance contained in it.

For details of the operating mode of a dose setting mechanism with a forward feed sleeve or a forward feed element, reference may be made to German patent application number 10 2004 041 151.4 and corresponding US application 2007/0197975 by the present applicant, the disclosures of which, including the disclosure relating to the design of a dose setting mechanism and the co-operation of flexible elements bearing cams or lugs and mounted on a forward feed sleeve and an outer sleeve or housing so as to co-operate with a toothed rack, are incorporated in this application by reference.

As regards the at least one thread provided in the internal and external face of the rotating sleeve and having a different pitch, in some preferred embodiments, the pitch of the thread engaging with the forward feed sleeve is smaller than the pitch of the at least one oppositely lying thread which engages with the housing or another component of the injection device relative to which the forward feed sleeve is mounted so that it can not rotate. Thus, a rotation of the rotating sleeve relative to the housing, forcibly produced by a setting operation due to a thread engagement of the rotating sleeve with the housing, is not converted into a rotation of the forward feed element, which also sits in a thread engagement with the rotating sleeve, but causes an axial movement of the forward feed sleeve relative to the housing. Accordingly, the forward feed element maybe provided with an external or internal thread both inside and outside the rotating sleeve. It is also possible for the pitch of the thread with which the rotating sleeve ad forward feed element engage to be bigger than the pitch of the thread provided on the other side of the rotating sleeve, in which case a short setting movement is converted into a relatively longer axial movement of the forward feed element.

In some preferred embodiments, a setting element is provided on the rotating sleeve, for example a control knob, which may be fixedly connected to the rotating sleeve so that the rotating sleeve can be screwed out of the injection device by rotating the control knob. The rotating sleeve may also be rotatably connected to the setting element or control knob so that the control knob can be pulled out of the housing of the injection device without a rotating movement, and the rotating sleeve rotatably mounted in the control knob is screwed out of the housing of the injection device.

In some preferred embodiments, the threads provided on the rotating sleeve are of the type not retained by friction, and the pitch angle of the thread may be selected so that the tangent of this pitch angle is greater than the coefficient of friction of the adjacently lying materials involved in the thread engagement. Lubricating materials such as Teflon, for example, may also be used to produce thread engagements that are not retained by friction. It would likewise be possible to provide a torsion spring which is tautened or tensed when the rotating sleeve is extracted or rotated and which is connected to the rotating sleeve so that when the rotating sleeve is pushed in or screwed in, the spring force acts in the direction of rotation, in which case it is also possible to use threads retained by friction and the friction of the threads is overcome by the spring.

In some preferred embodiments, the external and internal thread of the rotating sleeve are disposed coaxially with one another, i.e. the threads overlap in the axial direction, in which case a reduction or increase in ratio can be achieved by a compact design in which the injection device is of a shorter length.

In some preferred embodiments, radial and/or axial stops may be provided which restrict the movement of the forward feed element and/or the rotating sleeve in the radial and/or axial direction relative to the housing of the injection device. Two stops may be provided on the forward feed element, spaced apart from one another in the axial direction for example, which co-operate with a stop element of the housing so that the forward feed element can be moved in the axial direction of the injection device relative to the housing but only by a predefined distance d, in which case a dose quantity which can be dispensed by the injection device can be set which corresponds to the distance d. Radial stops may also be provided on the rotating sleeve and/or on the elements co-operating with the rotating sleeve, in other words the forward feed element and another element such as the housing, which restrict the rotating movement of the rotating sleeve and which permit a maximum rotation of, for example, only 180° or two fall rotations. A fixed dose maybe set by such stops, in which case a user pulls the setting element out as far as a stop of an element, and the preset dose is dispensed when the setting element is pushed in.

In some preferred embodiments, a marking is provided on the rotating sleeve and/or a setting element connected to the rotating sleeve, such as a control knob, for example, from which a reading can not be taken when in the state pushed into the injection device or from which a reading can be taken through an orifice or transparent material, for example, and which can be seen or read by a user when the rotating sleeve and/or a control element connected to the rotating sleeve is pulled out of the injection device or the housing of the injection device. Thus, a user may read the fixed dose set on the basis of the extraction movement which is then dispensed from the injection device by the pushing-in movement transmitted to the toothed rack and the stop.

In some embodiments, the present invention further relates to an injection device containing a substance to be dispensed or in which an ampoule can be inserted, and which has a dose setting mechanism of the type described above which can be coupled with a plunger body or plug of the injection device so that a dose to be dispensed can be set by means of the rotating sleeve and administered.

### BRIEF DESCRIPTION OF THE DRAWINGS

FIG. 1, including FIGS. 1A to 1C, illustrates a dose setting mechanism from German patent application 10 2005 001 159.4 in an initial position, during setting and after dispensing a dose;

FIG. 2, including FIGS. 2A and 2B, shows a cross-section through the dose setting mechanism of FIG. 1;

FIG. 3 illustrates an injection device with a dose setting mechanism of the type disclosed in German patent application 10 2005 001 159.4; and

FIG. 4, including FIGS. 4A-4C, illustrates an embodiment of an injection device in accordance with the present invention in an initial position, in a primed state and after dispensing; and

FIG. 5 is a view in cross-section along line 5-5 of FIG. 4.

### DETAILED DESCRIPTION

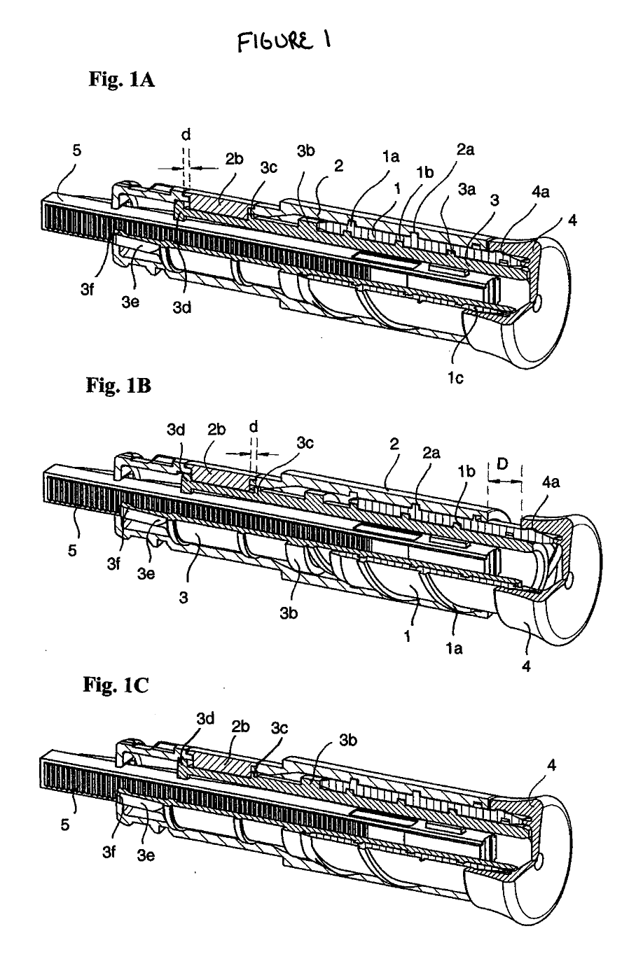

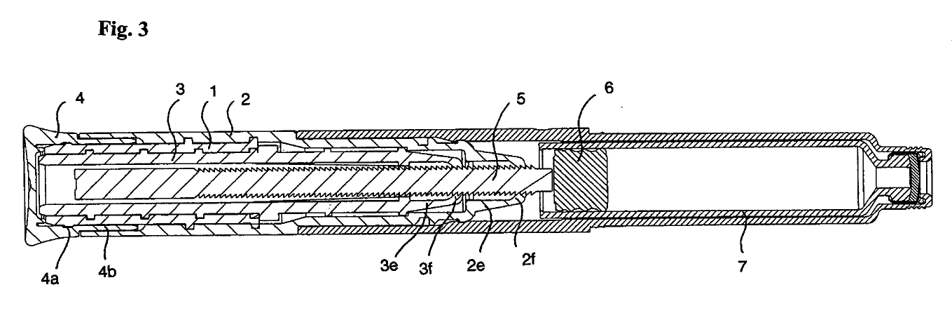

FIGS. 1A to 1C illustrate a dose setting mechanism of the type disclosed in German patent application 10 2005 001 159.4 for an injection device such as that illustrated by way of example in FIG. 3, with a housing 2 of the injection device, which has an internal thread 2a with a first pitch of 49°, for example. Mounted coaxially in the housing so as to be rotatable is a rotating sleeve 1 with an external thread 1a with the same pitch as the internal thread 2a of the housing 2, which engages in the internal thread 2a. The rotating sleeve 1 has an internal thread 1b with a pitch of 34°, for example, which is smaller than the pitch of the external thread 1a and the internal thread 2a of the housing, The difference in thread pitches is in the range of 10 to 15 degrees, for example.

Mounted inside the rotating sleeve 1 so as to be prevented from rotating relative to the housing 2 is a forward feed element 3, which has an external thread 3awhich engages in the internal thread 1b of the rotating sleeve 1. On the distal or front end of the forward feed element 3, illustrated on the left-hand side of FIG. 1A, are two oppositely lying cams or lugs 3f attached to elastic arms 3e, which are able to engage in the teeth on the external face of a toothed rack 5, which is disposed so that it is able to slide axially inside the forward feed element 3. The lugs or cams 3f of the forward feed element 3 as well as the lugs or cams 2f joined to the housing 2 and illustrated in FIG. 3 can be moved in the proximal direction relative to the toothed rack, in which case the lugs 2f, 3f are pushed outwardly against the elastic or spring force of the elastic arms 2e respectively 3e so that they can be moved past one or more teeth of the toothed rack 5. However, the lugs 2f and 3f are pushed toward the toothed rack 5 by the forward biasing action of the elastic arms 2e and 3e acting in the direction towards the toothed rack 5, they thus engage with the teeth so that the lugs 2f and 3f are not able to move in the distal direction of the toothed rack 5 and are prevented moving due to the lugs 2f and 3f engaging in the teeth of the toothed rack 5.

At the proximal end, the rotating sleeve 1 is connected to a control knob 4 and is mounted so as to be rotatable in it by a circumferentially extending groove 4a provided on the internal face of the control knob 4, in which a circumferentially extending ring 1c provided on the external face of the rotating sleeve 1 engages. When the control knob 4 is pulled out of the housing 2 by a user, the rotating sleeve 1 is driven with it and is rotated relative to the housing 2 as it is pulled out due to the external thread 1a engaging in the internal thread 2a of the housing 2. This rotation of the rotating sleeve 1 is converted into an axial movement of the forward feed element 3 due to the external thread 3a of the forward feed element 3, mounted so that it is not able to rotate relative to the housing 2, which engages in the internal thread 1b of the rotating sleeve 1. Since the pitch of the external thread 1a of the rotating sleeve is bigger than the internal thread 1b of the rotating sleeve, an outward movement D, indicated in FIG. 1B, is converted into a shorter outward movement d of the forward feed element 3 so that small, precise dose quantities can be set.

Provided on the external face of the forward feed element 3 are two axially spaced-part stops 3c and 3d projecting radially outwardly, between which the stop element 2b connected to the housing 2 engages. In the initial position illustrated in FIG. 1A, the distal axial stop 3c of the forward feed element lies against the distal side of the stop element 2b. The proximal stop 3d of the forward feed element is at a distance d from the distal side of the stop element 2b.

FIG. 1B illustrates the dose setting mechanism of the present invention after pulling out the control knob 4 by a distance D of 5 mm, for example, which causes the reduced axial movement by the distance d of 0.8119 mm of the forward feed element 3, for example, until the stop 3d of the forward feed element 3 lies against the distal side of the stop element 2b, thereby restricting the outward movement of the control knob 4. The rotating sleeve has been turned by −90°, for example.

When the control knob 4 is pulled out and the forward feed element 3 moved in the proximal direction, the lugs or cams 3f joined to the forward feed element 3 are pushed in the proximal direction along the toothed rack 5 retained by the lugs 2f joined to the housing 2, so that the oppositely lying lugs 3f are pushed backwards across one, two or more teeth of the toothed rack 5, for example.

When a user pushes on the control knob 4 and pushes it back into the housing 2, as illustrated in FIG. 1C, the pushing-in movement of the control knob is transmitted to the rotating sleeve 1, which rotates by +90° relative to the housing 2 due to the thread engagement with it, for example, and the thread engagement with the forward feed element 3 causes the latter to move axially forward until the proximal stop 3c is lying against the stop element 2b again and the front end of the rotating sleeve 1 is lying against a stop 3b of the forward feed element 3. As this happens, the toothed rack 5, retained by the lugs 3f engaging in the teeth, is pushed together with the forward feed element 3 in the distal direction so that the toothed rack is pushed by the distance d relative to the lugs 2f joined to the housing 2 which engage in teeth of the toothed rack 5 at the end of the forward movement and are axially offset in the proximal direction from the initial position illustrated in FIG. 1A. This forward movement of the toothed rack 5 is transmitted to the plunger 6 illustrated in FIG. 3, which is pushed into the ampoule 7 inserted in the injection device and thus forces a substance, for example insulin, contained in the ampoule 7 so that the quantity of substance corresponding to the forward movement d of the plunger 6 is dispensed from the ampoule 7.

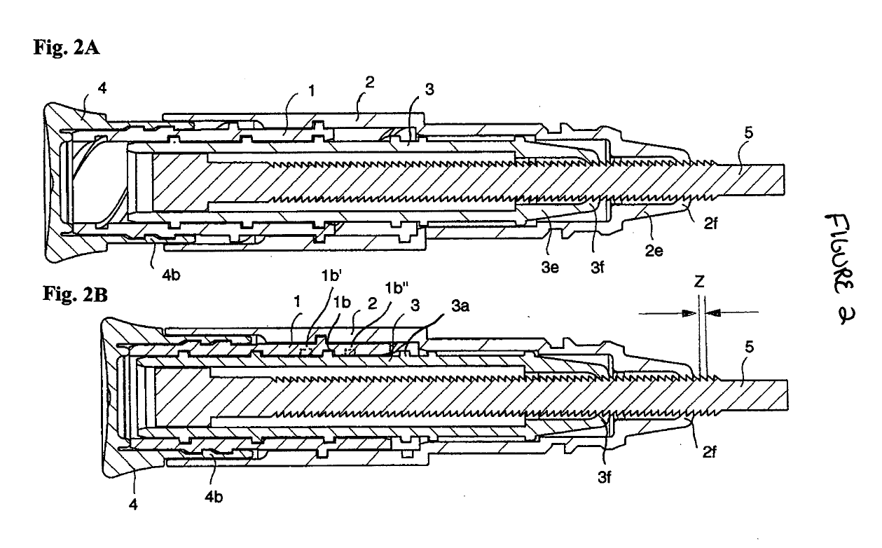

FIGS. 2A and 2B illustrate a cross-section through a different embodiment of the dose setting mechanism from German patent application 10 2005 001 159.4, in which the control knob 4 has webs 4b projecting axially into the housing 2 or alternatively a cylindrical, circumferentially extending element 4b which can be pushed into a co-operating recess of the housing 2. When the control knob 4 is pulled out, it is not the external face of the rotating sleeve 1 which is visible as illustrated in FIG. 1B for example, but the external face of the element 4b as illustrated in FIG. 2A.

Markings are provided both on the external face of the rotating sleeve 1 and on the external face of the element 4b, as a means of providing a user with information about a dose set due to the outward movement from the housing 2, displayed by coloured markings or rings, for example.

As may be seen from FIGS. 2A and 2B, for example, if a manufacturing method which results in manufacturing tolerances in the region of the distance Z of two teeth of the toothed rack 5 is used, a problem can arise whereby, when the dose setting mechanism is in the pushed-in state illustrated in FIG. 2B, for example, the teeth of the lugs 3f do not engage in the spaces between the teeth of the toothed rack 5 retained by the lugs 2f and instead lie on the teeth of the toothed rack 5. As a result of the present invention, a forward feed element 3 manufactured with a greater tolerance can be used if another internal thread in the form of one or more circumferentially extending grooves 1b′ or 1b″ is provided axially offset from the internal thread 1b of the rotating sleeve 1 provided in the form of the circumferentially extending groove, into which the external thread 3a of the forward feed element 3 formed by a circumferentially extending web can be screwed when the dose setting mechanism is being assembled so that, because of the resultant axial offset of the forward feed element 3, the teeth of the lugs 3f engage in the spaces between the teeth of the toothed rack 5 when the dose setting mechanism is again in the forward position illustrated in FIG. 2B.

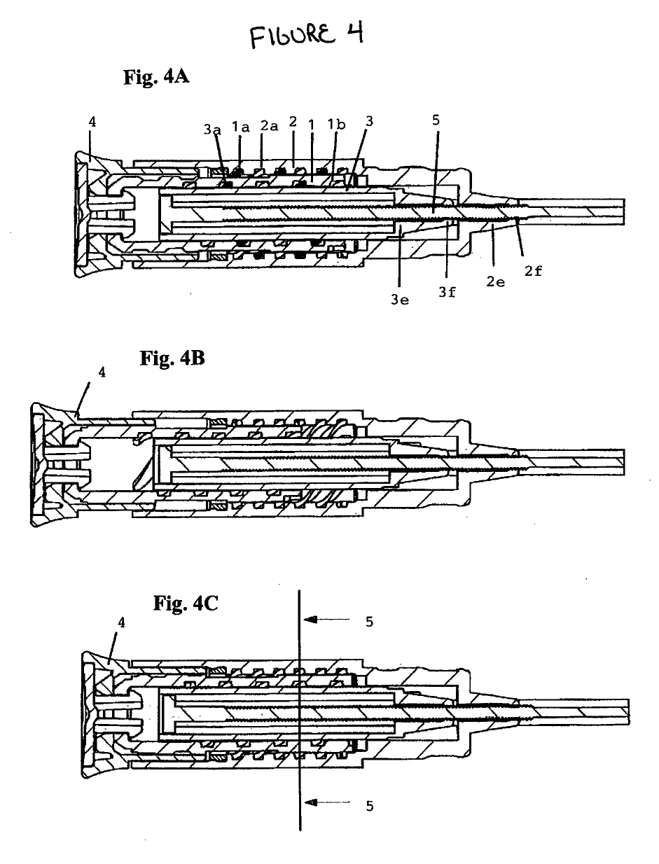

FIGS. 4A to 4C illustrate the rear end of an injection device in accordance with the present invention in an initial position, in a primed state and after dispensing from an ampoule 7 illustrated in FIG. 3. As may be seen from the threads illustrated in black, the external threads 1a and 3a of the rotating sleeve 1 and the forward feed element 3 provided in the form of circumferentially extending webs are guided in only one of a plurality of co-operating grooves forming the internal threads 1b and 2a of the rotating sleeve 1 and the housing 2, so that when the injection device is assembled, manufacturing tolerances of the housing 2, rotating sleeve 1 and/or forward feed element 3 can be compensated, thereby ensuring that the teeth of the snappers 2f and 3f always engage in spaces between the teeth of the toothed rack 5 in desired front and rear positions of the rotating sleeve 1 coupled with the knob 4.

As described above, the snappers 2f of the housing 2 and the snappers 3f of the forward feed sleeve 3 engage in the teeth of the toothed rack 5 and block it alternately as the knob 4 is pulled out and pushed in, so that the toothed rack 5 is moved in the distal direction only. As this happens, the rotating sleeve 1 couples the housing 2 and the forward feed sleeve 3 by a first thread between the housing 2 and the rotating sleeve 1 and a second thread between the rotating sleeve 1 and the forward feed sleeve 3. The threads may be of differing pitches.

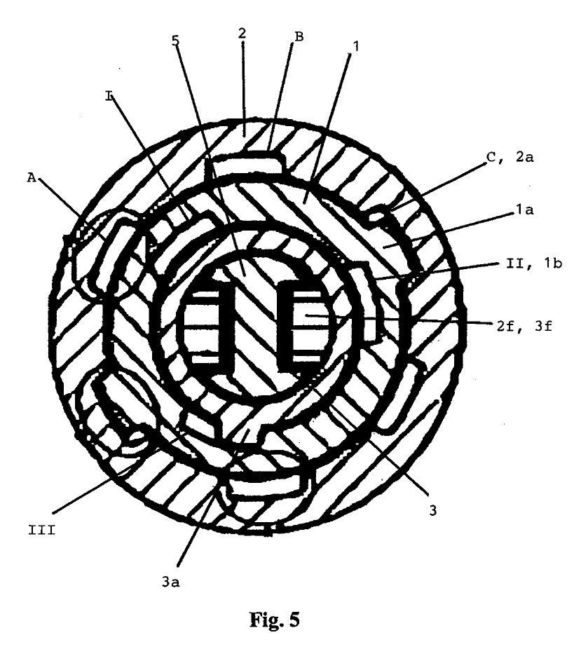

As illustrated in FIG. 5, one of several threads A, B or C provided in the housing 2 may be selected as the first thread between the housing 2 and the rotating sleeve 1. The threads A, B and C are disposed in the housing 2, distributed at approximately 120° but not exactly 120°, for example.

The different threads I, II and III are used to correct or adjust the orientation of the snappers 3f relative to the toothed rack 5, for example, so that the snappers 3f always fit in guide grooves provided along the toothed rack and in which the rows of teeth of the toothed rack 5 are disposed. When one of the threads I, II or II is selected for the engagement of the external thread 3 a of the forward feed sleeve 3, the forward feed sleeve 3 and the rotating sleeve 1 form a unit, the length of which can be varied by the position of rotation of the sleeves 1 and 3 relative to one another. To insert this unit in the housing 2, the external thread 1a of the rotating sleeve 1 is inserted in one of the threads A, B or C. If no length compensation is needed for the unit comprising the forward feed sleeve 3 and rotating sleeve 1 to enable the snappers 3f to engage fully in the rows of teeth of the toothed rack 5, i.e. a compensation due to manufacturing tolerances is not needed, the external thread 1a is inserted in thread C in the example described above where the thread has an offset of 118 degrees/122 degrees.

If it is necessary to correct the length of the unit comprising the forward feed sleeve 3 and rotating sleeve 1 for the snappers 3 f to engage fully in the rows of teeth of the toothed rack 5, the external thread 1a is inserted in thread B or C of the housing 2. To this end, the rotating sleeve 2 is rotated relative to the housing until the thread start of the external thread 1a lies opposite the thread inlet of thread B or C so that the external thread 1a can engage in thread B or C. As the rotating sleeve 1 is rotated, the forward feed sleeve 3 does not rotate with it relative to the housing 2, so that the rotation of the rotating sleeve 1 causes a change of length in the unit comprising forward feed sleeve 3 and rotating sleeve 1 and hence changes the position of the snappers 3f relative to the toothed rack 5, which remains stationary relative to the housing, as the forward feed sleeve 3 is pushed axially forwards or backwards.

One of several threads I, II or III provided in the rotating sleeve 1 may also be selected as the second thread between the rotating sleeve 1 and the forward feed sleeve 3, and the threads I, II and III may be distributed at exactly 120° around the rotating sleeve, for example.

The thread A, for example, is used to effect a negative length correction, thread B a positive length correction and thread C a zero correction. Zero correction is intended to mean that no tolerance correction is necessary. To this end, thread A is disposed offset by 118 degrees and thread B by 122 degrees from the thread C on the circumference of the housing 2. Naturally, it would also be possible to opt for a different combination of offsets, such as 119 degrees or 121 degrees, for example.

By an appropriate selection of a thread combination, the distance of the snappers 2f and 3f from one another by reference to the tooth spacing Z of the teeth of the toothed rack 5 can be adapted during assembly of the injection device so that all the teeth of the snappers 2f and 3f are able to engage fully in the teeth of the threaded rod 5 except during priming and pushing in movements, thereby enabling manufacturing tolerances to be compensated.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A setting mechanism for setting an injection device for administering a substance, comprising a first element and at least one other element displaceable relative to the first element, the first element having at least two mutually offset threads so that at least one thread element of the at least one other element is guided in only one of the at least two mutually offset threads.

2. The setting mechanism as claimed in claim 1, wherein the first element is one of a rotating sleeve, a housing or a forward feed element associated with the injection device, and the at least one other element is another one of the rotating sleeve, housing or forward feed element.

3. The setting mechanism as claimed in claim 1, wherein the at least two mutually offset threads of the first element are at least one of radially and axially offset from one another.

4. The setting mechanism as claimed in claim 3, wherein the at least two mutually offset threads of the first element are in the form of one of an internal thread or external thread on the first element.

5. The setting mechanism as claimed in claim 1, wherein the at least two mutually offset threads of the first element are in the form of circumferentially extending grooves or groove portions, and the at least one thread element of the at least one other element is in the form of a circumferentially extending web or web portion.

6. The setting mechanism as claimed in claim 1, wherein the first element has n threads for guiding m thread elements of the at least one other element wherein at least one of n>m≧1 and m•k=n, where k is a natural number.

7. A dose setting mechanism with at least one setting mechanism comprising a first element and at least one other element is mounted to be displaceable relative to the first element, the first element having at least two mutually offset threads so that at least one thread element of the at least one other element is guided in only one of the at least two mutually offset threads, wherein the at least two mutually offset threads are in the form of circumferentially extending grooves or groove portions and the at least one thread element of the at least one other element is in the form of a circumferentially extending web or web portion, and wherein a web of a thread of the at least one other element engages with one of the grooves of the first element so that a predefined positional relationship is achieved between the first element and the other element.

8. The dose setting mechanism as claimed in claim 7, wherein the positional relationship of the first element relative to the other element is predefined by the relative position of at least one engaging element or snapper on the first element or the other element.

9. A method of manufacturing a setting mechanism for an injection device, whereby a thread of at least one dose setting element is inserted in one co-operating thread of at least two threads of another dose setting element so that a predefined positional relationship is obtained between the dose setting element and the other dose setting element.

10. A dose setting mechanism for an injection device, the dose setting mechanism comprising a setting element, an approximately cylindrical rotating sleeve connected to the setting element and an external thread and an internal thread which has the same pitch as or different pitch from the external thread, wherein one thread of the rotating sleeve engages with a thread associated with one of the injection device or a component thereof, and the other thread engages with a thread of a forward feed element so that a setting movement of the setting element is transmitted by the rotating sleeve via the two thread engagements to produce a dose setting movement of the forward feed element, and wherein at least one of the external thread and the internal thread comprises at least two different grooves extending circumferentially around the rotating sleeve.

11. The dose setting mechanism as claimed in claim 10, wherein the pitch of the thread of the rotating sleeve which engages with the forward feed element is one of larger or smaller than the pitch of the other thread of the rotating sleeve.

12. The dose setting mechanism as claimed in claim 11, wherein the difference in the thread pitches is in the range of one of approximately 5 to 30 or approximately 10 to 15 degrees.

13. The dose setting mechanism as claimed in claim 10, wherein the injection device comprises a housing, and the forward feed element is mounted so that it is prevented from rotating relative to the housing.

14. The dose setting mechanism as claimed in claim 13, wherein at least two internal threads are provided on the housing, and the external thread of the rotating sleeve engages in at least one but not in all internal threads and the forward feed element has at least one external thread which engages in at least one of several internal threads of the rotating sleeve.

15. The dose setting mechanism as claimed in claim 14, wherein the rotating sleeve is one of fixedly or rotatably connected to the setting element.

16. The dose setting mechanism as claimed in claim 14, wherein the thread pitches of the rotating sleeve, the housing and the forward feed element are selected so that the threads are not retained by friction.

17. The dose setting mechanism as claimed in claim 10, further comprising a spring element which acts in or opposite the direction of rotation of the rotating sleeve.

18. The dose setting mechanism as claimed in claim 10, wherein the external thread of the rotating sleeve is disposed coaxially with and at least partially overlapping the internal thread of the rotating sleeve in a radial direction.

19. The dose setting mechanism as claimed in claim 10, further comprising a catch element on the forward feed element, said catch element carried on an elastic arm.

20. The dose setting mechanism as claimed in claim 13, further comprising a toothed rack axially displaceably inside the dose setting mechanism and inside the forward feed element, the rack having at least one or two rows of teeth on an external face in which one of an elastically mounted catch lug carried on the forward feed element or an elastically mounted catch lug joined to the housing can engage.

21. The dose setting mechanism as claimed in claim 13, further comprising at least one radial or axial stop on the forward feed element, the rotating sleeve or on the housing to restrict at least one of a radial or axial movement of the one of the forward feed element or the rotating sleeve relative to the housing, a positional relationship between the rotating sleeve, the housing or the forward feed element being defined by the stop.

22. The dose setting mechanism as claimed in claim 10, wherein markings are provided on at least one of an external face of the setting element and an external face of the rotating sleeve to display a set dose.

23. An injection device in which a substance to be dispensed is contained or in which an ampoule containing a substance to be dispensed can be inserted, the injection device comprising a setting mechanism comprising a first element and at least one other element displaceable relative to the first element, said first element having at least two mutually offset threads and said at least one other element having at least one thread element, whereby said at least one thread element of the at least one other element is guided in only one of said at least two mutually offset threads of the first element.
